(19) United States
(12) Patent Application Publication
(10) Pub. No.: US 2013/0165771 A1
(43) Pub. Date: Jun. 27, 2013

(54) USE OF FITC-DEXTRAN AS DYE FOR FUNDUS ANGIOGRAPHY AND METHODS THEREOF

(71) Applicants: Ming Ni, Laguna Niguel, CA (US); Kun Xu, Laguna Niguel, CA (US); Jun Lin, Scarsdale, NY (US)

(72) Inventors: Ming Ni, Laguna Niguel, CA (US); Kun Xu, Laguna Niguel, CA (US); Jun Lin, Scarsdale, NY (US)

(21) Appl. No.: 13/716,780

(22) Filed: Dec. 17, 2012

Related U.S. Application Data
(60) Provisional application No. 61/577,009, filed on Dec. 17, 2011.

(51) Int. Cl. A61B 3/12

(52) U.S. Cl. CPC A61B 3/1241
USPC 600/431

(57) ABSTRACT

A novel fundus angiographic application and method using fluorescein isothiocyanate dextrans (FITC-dextran) as fluorescence angiographic dye for diagnosis and evaluation of eye diseases on humans is disclosed. The method involves the process of constitution of FITC-dextran solution and intravenous injection of the said solution, followed by observation and photography of ocular fundus circulation using a fluorescence fundus camera with an image system. The invention offers improved fundus angiograms over the traditional Na-fluorescein fundus angiography (Na-fluorescein) and indocyanine green fundus angiography (ICG) with the advantages of higher clarity and higher resolution; capable of visualizing both the retinal and the choroidal circulations systems simultaneously; providing longer fundus image duration so that it can be used as a directly mapping guidance in retinal surgery or to complete the angiograms for both eyes by a single process; providing more information about the pathological changes occurred among retinal, sub-retinal and choroidal circulation systems.

### CROSS-REFERENCE TO RELATED APPLICATION

The present invention claims the priority benefit under 35 U.S.C. §119(e) of U.S. Provisional Application No. 61/577,009, filed Dec. 17, 2011, the entire disclosure of which is incorporated herewith by reference.

### FIELD OF THE INVENTION

The invention relates to fluorescence angiography in ophthalmology, in particular, to a novel diagnostic application and method using fluorescein isothiocyanate dextran (FITC-Dextran) as dye in fundus angiography for visualization and evaluation of ocular circulations in normal or diseased eyes in humans or other animal species.

### BACKGROUND OF THE INVENTION

Fundus fluorescein angiography, also called fundus angiography is a conventional clinical diagnostic technique used to visualize the vasculature of the fundus for the purposes of diagnosis of the ocular diseases. The general procedure of a fundus angiography consists of administering the dye by intravenous injection and photographing the fluorescent image of the fundus with a fundus camera to obtain angiograms of vascular circulations in the eye. The pathological changes in the eye, such as neovascularization, blockage or leakage of blood vessels, and hemorrhage, can be visualized and recorded by fundus angiography which makes the fundus angiography an important tool for ophthalmologists. Currently, there are two dyes which are available for the fundus angiography in the clinical practice: sodium fluorescein (Na-fluorescein) and indocyanine green (ICG).

### Sodium Fluorescein Fundus Angiography

Sodium fluorescein fundus angiography is the fundus angiography which uses sodium fluorescein (Na-fluorescein) as the dye. Na-fluorescein is the salt form of fluorescein. Na-fluorescein molecules respond to light spectrum at wavelength between 465 and 490 nanometers (nm) and emit fluorescence at wavelength of 520 to 530 nm. The excitation wavelength is blue while the resulting fluorescence is green-yellow. Na-fluorescein has been used as dye in fundus angiography in human since 1950s (Ringvold et al. 1979; Schatz, et al. 1978; Yannuzzi, 1989).

In a typical process of Na-fluorescein fundus angiography, Na-fluorescein dye is prepared as an injectable solution and rapidly injected into antecubital vein or other intravenous accesses. In the blood stream, 70-80% of fluorescein molecules bind to serum proteins. The protein-bounded fluorescein molecules do not fluorescence. Only the free fluorescein (unbounded) molecules respond to light energy and emit fluorescence. Following blood stream the fluorescein molecules enter ocular circulation by ophthalmic artery, which supplies both choroid via the short posterior ciliary artery and retina via central retinal artery. The fundus angiograms are obtained with a fluorescence fundus camera by photographing the fluorescence emitted after illumination of the retina at a wavelength of 490 nanometers.

In choroid the free fluorescein molecules readily diffuse through the choriocapillaries. However, the free fluorescein molecules can't readily pass through the normal retinal capillaries because the tight junction of the endothelium cells, e.g., the blood-retina barrier. The leakage of Na-fluorescein molecules through choriocapilliaries results in the poor fluorescent image of choroidal vasculature from Na-fluorescein angiography. Therefore, Na-fluorescein angiography is mainly used for retinal angiography, such as in the cases of diabetic retinopathy, retinal vein or arterial occlusion or leakage, rather than choroidal vasculature angiography.

Because of the poor choroidal vasculature images, the fundus angiograms of Na-fluorescein is unable to provide satisfactory information for ophthalmologists for confirming the origin of abnormal blood vessels among retinal, sub-retinal, and choroid, which should be very meaningful for the diagnosis of ocular diseases. The poor image resolution of Na-fluorescein angiography also makes it impossible to provide morphological information about the detail of blood vessels especially under variety of pathological conditions.

Another limitation of Na-fluorescein fundus angiograph is that it unable to provide clear images on the area with the inflammation and/or neovascularization. Under such conditions, because the leakage or increase of the permeability of the capillaries, the focus is presented as a high density fluorescent spot on the Na-fluorescein angiograms without the details of the blood vessels.

The short effective image duration is another disadvantage of Na-fluorescein fundus angiography. Following intravenous injection of the dye the best effective image duration is about 5 minutes which makes the angiographic process inconvenience and difficulty to obtain satisfactory angiograms. The short effective image duration also makes it impossible to obtain the angiograms from both eyes of a patient in single fluorescein angiographic process.

### Indocyanine Green Chorioangiography (ICG)

ICG has been introduced into clinical practice ophthalmology as a dye for fundus angiography since 1993 (Guyer et al 1992; Yannuzzi, 1992). ICG fundus angiography is unable to provide retinal angiograms with the quality of Na-fluorescein angiography. However, ICG fundus angiography is able to visualize choroidal vasculature with better image quality than Na-fluorescein angiography, which makes ICG fundus angiography mainly used for visualizing the choroidal vasculature in clinical practice.

ICG is a cyanine dye which has a peak spectral absorption at about 800 nanometers (nm), emitting at 830 nm, both spectrums are within the infrared spectrum. In blood steam 98% of ICG molecules bound to serum proteins, which prevents the leakage of ICG molecules from choroidal capillaries, which is one of the reasons that makes ICG fundus angiography able to visualize choroidal vasculature.

Another nature of ICG fundus angiography is that, due to the long wavelengths and the relative low light emitting efficacy of ICG, the resolution and the contrast of the image between blood vessels and the surrounding tissues of ICG angiograms are not as good as the angiograms of Na-fluorescein. Thus, ICG fundus angiography is unable to provide adequate morphologic information about the pathological changes in the conditions such as retinal hemorrhage, pigment damages, or serosanguineous fluid, which make ICG angiography undesirable for the retina angiograms in the clinical practice (Bischoff et al, 1985).

To perform ICG fundus angiography, a special-infrared-frequency fundus image system equipped with a direct digital recording system with high speed image frame grabber is required. ICG has been reported with the toxic effects on both retinal pigmental endothelial cells and outer portion of the retinal tissues (Kodjikian, et al, 2005; Giuseppe, et al, 2008). Because of the limitations mentioned above and the adverse effects, the usage of ICG fundus angiography has been restricted

### SUMMARY OF THE INVENTION

In one aspect, the present invention provides a method for performing fundus angiography comprising administration of a fluorescein derivative-linked polymer as a fluorescence angiographic dye followed by observation and photography of ocular fundus circulation using a fluorescence fundus camera with an image system. The methodology of the application of FITC-dextran as dye for fundus angiography in human includes the constitution of FITC-dextran solution in pharmaceutical acceptable mediums, such as in injectable water, saline, PBS, or other types of buffer at desired concentration, the intravenous injection of the said FITC-dextran solution, following by observation and photography of the fundus images using a conventional fluorescein fundus image system.

In a preferable embodiment, the fluorescein derivative-linked polymer has a weight average molecular weight ranging from 3 k Daltons to 2000 k Daltons, such as 3 k, 5 k, 10 k, 20 k, 40 k, 70 k, 120 k, 250 k, 200 k, and 2000 k Daltons, preferably 40 k to 120 k Daltons, most preferably 70 k Daltons.

In a preferable embodiment, the fluorescein derivative-linked polymer is FITC-dextran. As it will be appreciated by the person skilled in the art, although the following description is made reference to one of the fluorescein derivative-linked polymer, i.e., FITC-dextran, the present invention is not limited to the exemplified polymer. Other fluorescein derivative-linked polymers can also be used as long as they meet the requirements specified or indicated in the present invention, such as fluorescein isothiocyanate dextrans (FITC-dextrans), NHS-fluorescein dextran, pentafluorophenyl esters dextran (PFP-dextran), Alexa 488 dextran, FluoProbes 488 dextran, DyLight 488 dextran, fluorescein isothiocyanate d-glucose polymer, fluorescein isothiocyanate chitin, fluorescein isothiocyanate chitin derivatives, and fluorescein isothiocyanate cellularglycan.

Preferably, each glucose unit of the dextrans is linked with one or more FITC moiety(s) through chemical linkage at C1, C2, C3, C4, and/or C6 carbon position of the glucose unit of the dextrans.

Prior to administration to a subject in need, the fluorescein derivative-linked polymer is dissolved at a concentration ranged from 0.1 wt. % to 50 wt. %, preferably from 0.1 wt. % to 10 wt. %, more preferably from 0.5 wt. % to 5 wt. % in water, saline, phosphate buffered saline (PBS) or other buffered systems with or without other chemical additive(s).

Preferably, the fluorescein derivative-linked polymer is administrated at a dose range of 0.1 to 100 mg/kg, preferably from 1 to 50 mg/kg through intravenous or other routes of administration based on doctor's instruction.

In another aspect, the present invention discloses a method for diagnosis of eye diseases comprising administration of a fluorescein derivative-linked polymer as a fluorescence angiographic dye to a subject in need of receiving fundus angiography, followed by observation and photography of ocular fundus circulation using a fluorescence fundus camera with an image system. Preferably, the eye diseases are selected from the group consisting of retinal, sub-retinal or choroidal neovascularization; proliferative diabetic retinopathy; retinopathy of prematurity; retinal tumors; degenerative conditions; ocular inflammation and optic nerve lesions; choroidal tumors; and retinal vascular occlusion.

In another aspect, the present invention discloses a method for qualitative or quantitative analysis of changes of ocular blood vessels comprising administration of a fluorescein derivative-linked polymer as a fluorescence angiographic dye to a subject in need of receiving fundus angiography, followed by observation and photography of ocular fundus circulation using a fluorescence fundus camera with an image system. Preferably, the eye diseases are selected from the group consisting of retinal, sub-retinal or choroidal neovascularization; proliferative diabetic retinopathy; retinopathy of prematurity; retinal tumors; degenerative conditions; ocular inflammation; optic nerve lesions; choroidal tumors; and retinal vascular occlusion.

The advantages of the present invention over the conventional Na-fluorescein fundus angiography or ICG chorioangiography include: (1) providing the fundus angiograms with high quality of clarity and high resolution; (2) being able to visualize both retinal vasculature and choroidal vasculature simultaneously; (3) providing longer effective image duration which allows to obtain both eye's angiograms from a single process, instead of one angiography process for only one eye; (4) providing more morphologic nature of the ocular diseases such as the pathological changes in retinal or retinal pigment epithelium (RPE) detachment or neovascularization; (5) being used as a tool for the evaluation of the vascular permeability in vascular disorders or inflammatory diseases; (6) providing directly mapping information used as a guidance for retinal surgery treatment purpose.

### BRIEF DESCRIPTION OF THE DRAWINGS

FIG. 1 shows the FITC-dextran fundus angiogram which visualizes both the retinal vasculature and choroidal vasculatures on the same angiogram. The angiograms are obtained from FITC-dextran fundus angiography from the eye of a naive Dutch-belted rabbit by using FITC-dextran with average molecular weight of 70 kDa as dye. Both the retinal vasculature (red arrows in A, B, C) and the choroidal vasculature (yellow arrows in A, B, C, D), are visualized with superior clarity and resolution on the angiogram.

FIG. 2 shows the choroidal angiograms obtained from normal rabbit eye by FITC-dextran fundus angiography. The angiograms of choroid were obtained within a 2-hour period following a single dose injection of FITC-dextran with the average molecular weight of 70 kDa. The images from A to H were captured at the time points of 10^{th} second, 30^{th} second, 1.0^{st} min, 1.4^{th} min, 10.4^{th} min, 32.6^{th} min, 59.3^{rd} min, and 119.4^{th} min post intravenous administration of FITC-dextran correspondently.

FIG. 3 shows the fundus angiograms of FITC-dextran from the eye with retinal detachment. Image A presents the fundus angiogram from the normal eye. Image B presents the angiogram from the eye with retinal detachment. The yellow arrows in image B indicate the area of the retinal detachment with clear images of the edges of the detachment, the fluid accumulated in the sub-retinal space of the detachment, and the choroidal vasculature beneath the retinal detachment area. The both images were taken from the same rabbit eye in a single angiography process.

FIG. 4 shows the angiograms obtained from the eye with choroidal neovascularization in rabbit using FITC-dextran fundus angiography. The images of A-OD and A-OS represent the fundus angiograms of both, the right eye and the left eye correspondently before CNV induction. The images of B-OD and B-OS present the fundus angiograms of both, the right eye and the left eye correspondently, with new blood vessels grown from choroidal. The red arrows indicate the areas with new blood vessels in the retina.

FIG. 5 shows the fundus angiograms of FITC-dextran Angiography by using FITC-dextrans with different average molecular weights as the dyes on the eye with choroidal neovascularization in rabbits. The degrees of the leakage of FITC-dextran from choroidal blood vessel were found to be molecular weight dependent among FITC-dextran molecular weights of 4 kDa, 20 kDa, 40 kDa, and 70 kDa (A, B, C, and D), with the FITC-dextran 4 kDa has the highest detective sensitivity for the leakage of the CNV among the tested FITC-dextrans. There is no apparent difference in the degree of the leakage among the angiograms obtained by using the FITC-dextrans with molecular weights of 70 kDa, 150 kDa, or 2000 kDa (D, E, and F).

FIG. 6 shows comparison of FITC-dextran fundus angiograms with Na-fluorescein fundus angiograms obtained from the eyes with choroidal neovascularization in the rabbits. FIGS.-6A and -6C show the images from Na-fluorescein fundus angiography on which the area with the choroidal new blood vessels was presented as a hard spot of fluorescein (red arrows) without the morphological details of the CNVs. FIGS.-6B and -6D show the images of FITC-dextran angiography on which the details of the newly grown vasculature were revealed which include the source of the newly grown blood vessels stemmed from choroid and the structures and the distributions of the newly grown blood vessels.

FIG. 7 shows comparison of FITC-dextran fundus angiograms with Na-fluorescein fundus angiograms obtained from the eyes with choroidal newly blood vessels growing through the fluid and the detached retina in the rabbits. FIGS.-7A and -7C show the images from Na-fluorescein fundus angiography. On the angiograms the area with newly grown blood vessels from choroid was present as a hard spot of fluorescein (red arrows) without the morphological details about the blood vessels and the detachment. FIGS.-7B and -7D show the images of FITC-dextran angiography on which the morphological nature of the newly grown vasculature and the detachment of the retina were visualized with the details of the structure and the distributions of the newly grown blood vessels, the size and the edges of the detached retinas, and the choroidal blood vessels underneath the detachment.

FIG. 8 shows comparison of FITC-dextran fundus angiography with Na-fluorescein fundus angiography on the eye with choroidal neovascularization complicated with the sub-retinal hemorrhage in rabbit. FIG.-8A shows the image of Na-fluorescein fundus angiography. On the angiogram the area with the choroidal new blood vessels was present as a hard fluorescein spot (red arrows) without the morphological details about the CNV and the hemorrhage. FIG.-8B shows the image of FITC-dextran fundus angiography which is able to provide the morphological nature of the newly grown vasculature (yellow arrows) and the details of the structure and the distributions of the newly grown blood vessels, and the area with the sub-retinal hemorrhage (asterisks).

FIG. 9 shows comparison of FITC-dextran fundus angiography with Na-fluorescein fundus angiography, and ICG-fundus angiography on the angiograms obtained from the eye with choroidal neovascularization complicated with proliferated retinopathy and atrophy of choroidal circulation in the rabbit. FIG. 9A shows the angiogram of Na-fluorescein angiography. On the angiogram, the area with CNV was present as a high density fluorescence spot (red arrows in 9A) without morphological details about the CNV and the proliferated retinopathy and the atrophy of choroidal vasculature. FIG.-9B presents the angiogram of ICG fundus angiography obtained from the same eye. ICG fundus angiography is able to visualize the structure of the CNV and the injured choroidal vascular networks (red arrows in 9B). FIG.-9C presents the angiogram of FITC-dextran obtained from the same eye. Comparing with ICG angiography or Na-fluorescein angiography, FITC-dextran angiogram is able to provide the details about the newly grown blood vessels with high clarity and resolution. In addition, FITC-dextran angiography is able to provide the image with the morphological details about the complicated proliferated retinopathy and the atrophy of the choroidal vasculature.

FIG. 10 shows comparison of FITC-dextran fundus angiography with Na-fluorescein fundus angiography on the angiograms from the eye with retinal neovascularization in monkey. FIG.-10A presents the fundus angiogram of Na-fluorescein angiography. The angiogram of Na-fluorescein angiography shows the area with the newly grown blood vessels as a high density fluorescence spot (arrows in 10A) without morphological details of the newly grown blood vessels. The effective image duration of Na-fluorescein fundus angiography is about two minutes flowing injection of the dye. FIG.-10B presents the angiogram of FITC-dextran fundus angiography from the same eye. FITC-dextran fundus angiogapgy is able to visualize the newly grown blood vessels with the details about the source of the newly grown blood vessels and its distribution. The angiogram of FITC-dextran also shows the spots of the leakages from the newly grown blood vessels. The effective image duration of FITC-dextran fundus angiography was maintained up to 22 minutes post the injection of FITC-dextran of 40 kDa.

FIG. 11 shows the fluorophotometry analysis using FITC-dextran on the eyes with ocular inflammation. Endotoxin-induced uveitis (EIU) was induced in pigmented rabbits by intravenous administration of 10 μg/kg lipopolysaccharide (LPS). The breakdown of the blood-aqueous barrier in the anterior (Peaks on right) and the posterior segment (peaks in left) of the eye were measured by fluorophotometry at the 90^{th} minutes post LPS challenge. FITC-dextran of 70 kDa was given (3 mg/kg, intravenously) at the 5^{th} minutes before fluorescence measurement. Fluorescence (AUC) indicates the degree of intraocular inflammation.

### DETAILED DESCRIPTION

### Definitions

### Fluorescein

Fluorescein is a synthetic organic compound available as a dark orange/red powder soluble in water and alcohol. It is widely used as a fluorescent tracer for many applications. Fluorescein is a fluorophore commonly used in microscopy, in a type of dye laser as the gain medium, in forensics and serology to detect latent blood stains, and in dye tracing. Fluorescein has an absorption maximum at 494 nm and emission maximum of 521 nm (in water). It also has an isosbestic point (equal absorption for all pH values) at 460 nm. It was first synthesized by Adolf von Baeyer in 1871. It can be prepared from phthalic anhydride and resorcinol in the presence of zinc chloride via the Friedel-Crafts reaction.

### Fluorescein Isothiocyanate (FITC)

There are many fluorescein derivatives, among which, fluorescein isothiocyanate, often abbreviated as FITC, is the original fluorescein molecule functionalized with an isothiocyanate group (—N═C═S), replacing a hydrogen atom on the bottom ring of the structure. The structure of FITC is shown by formula I.

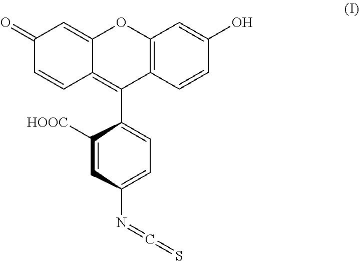

FITC responds to light energy between 465 and 490 nm emitting fluoresce at a wavelength of 520 to 530 nm. FITC has been used in wide-ranging applications in biology research area such as flow cytometry or protein labeling.

### Dextran

Dextran is a complex glycan with branches of various lengths. They are characterized by moderate to high molecular weights, good water solubility, low toxicity, and low immunogenicity. Depending on the length of side chains, dextran varies with different molecular weights from 3 to 2000 kilo Daltons. The straight chain consists of α-1,6 glycosidic linkages between glucose molecules, while branches begin from α-1,3 linkages. Dextran has been used medicinally as an antithrombotic (anti-platelet) to reduce blood viscosity or as a plasma volume expander in humans for over 50 years (Gronwall et al. 1957; Thorsen et al. 1950; Gelin et al. 1956). The structure of dextran is shown by formula II.

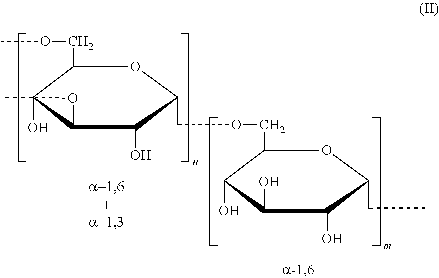

### FITC-Dextran

FITC-dextran is prepared by coupling special dextran fractions to fluoresceinyl isothiocyanate (5-isomer). In the FITC-dextran, each glucose unit of the dextrans is linked with one or more FITC moiety(s) through chemical linkage at C1, C2, C3, C4, and/or C6 carbon position of the glucose unit of the dextrans. The molecules of FITC-dextran are stable for at least 24 hours in vivo and have excellent biocompatibility. The substance of FITC-dextran presents as a yellow/orange powder which can be dissolved freely in water or salt solutions to form a yellow solution. FITC-dextran also can be dissolved in DMSO, formamide, and certain other polar organic solvents but is insoluble in lower aliphatic alcohols, acetone, chloroform, or dimethylformamide. The solubility of FITC-dextran in water is about 50 mg/ml. Excitation spectrum of FITC-dextran is best performed at 493nm and fluorescence measure at 518 nm.

FITC-dextran has been broadly used as a trace reagent in the variety of in vitro studies such as in microcirculation or membrane permeability studies in cells or tissues cultures. FITC-dextran has also been used in variety of animal researches for, such as, visualizing or quantifying the ocular neovascularization, evaluating inflammation response, or for retinopathy of prematurity (ROP) studies in mouse (Huang et al. 2010; Waterbury et al. 2006; Davies et al. 2003, 2006, 2008; McKenna et al. 2001, Ohno et al. 2003; Yu et al. 2009), rat (D'Amato et al. 1993; Baffi et al. 2000; Campos et al. 2006, Edelman et al. 2000; Fu et al 2007; Honda et al. 2011; Kinose et al. 2005; Olson et al. 2007, 2009; Semkova et al. 2003; Wang et al. 2007; Bee et al. 2010; Zou et al. 2007; Rabkin et al. 1977), or for the studies of neovascularization (CNV) in chicken horioallantoic membrane (Patrycja et al 2011), or in mouse (Barreiro et al. 2003; Campa et al. 2008; Feeney et al. 2003; Funakoshi et al. 2006, Hu et al. 2008; Ida et al. 2003; Tobe et al. 1998), or rabbits (McNaught et al. 1981,) and monkey (Lightman et al. 1987 Miller et al 1994, Tolentino et al. 2000). In one study, FITC-dextran was employed as a research tool to measure the leakage of the choroidal vasculature in patients with uveitis (Atkinson et al 1991). In this study, Na-fluorescein or FITC-dextrans with molecular weights of 4 KDa, 20 Kda, and 150 kDa were used as tracer to evaluate the permeability of choroidal vasculature in the eye with uveitis by using a conventional fluorescein fundus camera.

### Polymer

A polymer is a chemical compound or mixture of compounds consisting of repeating structural units created through a process of polymerization.

Polymers that are suitable for use with the present invention are polymer that suitable for use in living animals (e.g. human) in vivo, including homopolymers and copolymers. Preferably, homopolymers are used. According to the present invention, the polymer to be linked with a fluorescein derivative has a weight average molecular weight ranging from 3 k Daltons to 2000 k Daltons. Preferable polymers include dextran, d-glucose polymer, chitin or its derivatives, and glycan, e.g., cellular glycan.

In addition to the requirement of molecular weight, the polymer used in the present invention is linkable to a fluorecein derivative provided by the present invention, such as NHS-fluorescein, pentafluorophenyl esters, Alexa 488, FluoProbes 488, DyLight 488, and so on. The link is preferably embodied as covalent linkage.

As a last requirement for the polymers used in the present invention, when linked to a fluorecein derivative to form the fluorescein derivative-linked polymer, they should be substantively non-toxic at least to animal eyes, such as human eyes. Preferably, those polymers have no significant irritation or undesirable effect to human body.

As stated above, currently there are two kinds of fundus angiography technologies available for clinical fundus angiography: sodium fluorescein (Na-fluorescein) fundus angiography and indocyanine green (ICG) fundus angiography. Na-fluorescein is the dye used for sodium fluorescein fundus angiography and ICG is the dye used for ICG fundus angiography. Although the fundus angiography has significantly improved the diagnosis accuracy and provides meaningful guidance for the treatment of ocular diseases, the limitations of these two angiographic technologies make the fundus angiograph far from the satisfaction of clinical requirements.

In the present invention we disclose a method of using a fluorescein derivative-linked polymer (such as FITC-dextran) as dye for fundus angiography in human or other animals. The methodology of the application of FITC-dextran as dye for fundus angiography in human includes the constitution of FITC-dextran solution in pharmaceutical acceptable mediums, such as in injectable water, saline, PBS, or other types of buffers at desired concentration, and the intravenous injection of the said FITC-dextran solution, following by observation and photography of the fundus images using a conventional fluorescein fundus image system.

The superior advantages of FITC-dextran fundus angiography over the traditional angiographic technologies are that the novel fundus angiography provides better clarity and higher temporal resolution of the ocular angiograms than the angiograms from either Na-fluorescein angiography or ICG angiograph. In addition, the invention makes it possible to visualize both retinal vasculature and choroidal vasculature by single fundus angiographic process. With the extended image duration provided by FITC-dextran fundus angiography the fundus angiograms from both eyes of one patient can be obtained by a single angiography process. FITC-dextran angiography can also be used as a direct mapping guidance to localize the focus for therapeutic interventions such as surgery or laser, which not only is able to improve treatment results, but also to minimize the damages of the normal retina tissues caused by the therapeutic intervention process.

Although FITC-dextran has been broadly used in a variety of in vitro or in vivo experiments as mentioned above, there has never been a previous report about using FITC-dextran as an ocular fundus angiography dye for clinical application in ophthalmology on humans. It is the first time in the present invention discloses such novel application and method.

In normal conditions retina is nourished by two circulation systems. The central retinal artery supports nutrition to the inner two-thirds of the retina. The choroidal vasculature supports the outer one-third of the retina which includes retinal pigment epithelium layer (RPE), rods and cones photoreceptors. In blood stream, the free Na-fluorescein molecules readily diffuse through the choriocapillaris, however, the free fluorescein molecules do not diffuse through the normal retinal capillaries because the tight junction of the endothelium of the capillaries. Thus, Na-fluorescein fundus angiography is able to visualize retinal vasculature with clean image but not choroidal vasculature. FITC-dextran, on the other hand, diffuses through neither the normal retinal vasculature nor the normal choroidal vasculatures because of the large molecular weights (4 kDa to 200 Kda). Therefore the high concentration of FITC-dextran can be maintained not only in retinal vasculature, but also in the choroidal vasculatures, which makes FITC-dextran fundus angiography capable of visualizing both the retinal vasculature and choroidal vasculature simultaneously. In addition, the impermeability of FITC-dextran molecules results in better illumination contrast of the blood vessels against the surrounding tissues which consequently improves the clarity and the resolution of the angiograms. In certain pathological conditions, the leakage of FITC-dextran from blood vessel occurs since the increase of the permeability of capillary or breakdown of blood-retina barrier leakage. In such case the location(s) of the leakage can be precisely demonstrated based on the images of FITC-dextran angiography.

For a typical FITC-dextran fundus angiography, FITC-dextran dye is generally prepared as an injectable solution. The solution is injected rapidly (within about 10 seconds) into a convenient vein by a routine clinical procedure. Following the administration a series of the angiographic images of the fundus are taken according to the requirements of ophthalmologists.

The advantages of FITC-dextran angiography over Na-fluorescein angiography or ICG angiography include (1) being able to visualize both retinal vasculature and choroidal vasculature on same angiogram, instead of Na-fluorescein angiography for retinal vasculature and ICG angiography for choroidal vasculature, which provides more complete fundus information for the needs of ophthalmologist; (2) providing superior clarity and resolution of the fundus angiograms, so that the more detailed morphological information of the ocular can be visualized; (3) revealing more information about the morphological features of the abnormal vascular conditions of ocular diseases, such as neovascularization, retinal detachment, retinal hemorrhage, or hyper-pigment deposits; (4) indicating the precise location of the focus which can be used as an guidance for surgical treatment of ocular diseases so that the damage on the normal retina tissues caused by the surgery can be significantly reduced; (5) providing detailed information about the leakage of the blood vessels which can be used for the purposes of diagnosis of ocular disease or for the evaluation of therapeutic efficiency; (6) providing extensive effective image duration so that the angiograms from the both eyes can be performed by single angiography procedure, instead of two separate procedures, one for each eye. The advantage of the prolonged image duration of FITC-dextran fundus angiography also makes it possible to use it as the focus indicator for retinal laser treatment.

### EXAMPLES

The following examples illustrate aspects and embodiments of the invention and should not be constructed as limiting to the present invention.

### Example 1

FITC-dextran Fundus Angiography: Visualization of both Retinal Vasculature and Choroidal Vasculatures on the Same Fundus Angiogram.

Materials and Methods: The naive rabbits with bodyweight of 1.8-2.5 kilograms were used for this test. FITC-dextran with average molecular weight of 70 kDa (FD-70S, Sigma, St. Louis, Mo.) was used as the dye. The substance of FITC-dextran was dissolved in water to a concentration of 25 mg/ml. 0.3 ml of the FITC-dextran solution was injected intravenously to each rabbit. A regular fluorescence fundus system (Topcon 50 VT) was used for capturing the images of angiograms.

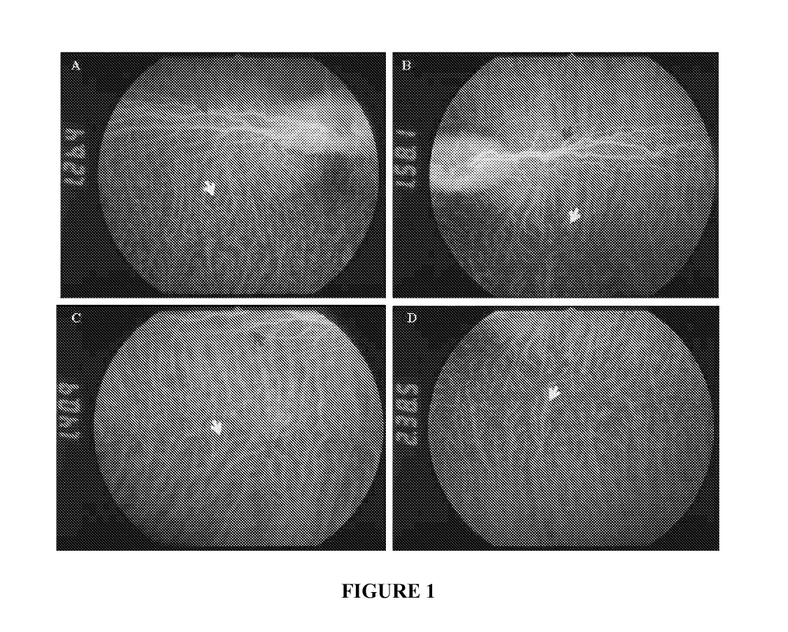

The results are shown in FIG. 1 which present the FICT-dextran fundus angiograms from normal rabbit eyes. The red arrows in FIGS. 1A, B, and C indicate the normal retinal vasculature and the yellow arrows in FIGS. 1A, B, C, and D indicate the normal choroidal vasculature, which demonstrate that the FITC-dextran angiography is able to visualize both retinal vasculature and choroidal vasculature in a same fundus angiogram.

### Example 2

### The Effective Image Duration of FITC-Dextran Fundus Angiography in Rabbit

Materials and Methods: FITC-dextran with average molecular weight of 70 kDa (FD-705, Sigma, St. Louis, Mo.) was dissolved in injection water at the final concentration of 25 mg/ml. The volume of 0.3 ml of the solution was injected through the marginal vein of a Dutch Belted rabbit. The fluorescein images of choroidal vasculature were monitored and captured up to 2 hours following the injection of FITC-dextran solution. The angiograms of choroidal vasculature were captured using a regular fluorescence fundus image system (Topcon 50 VT).

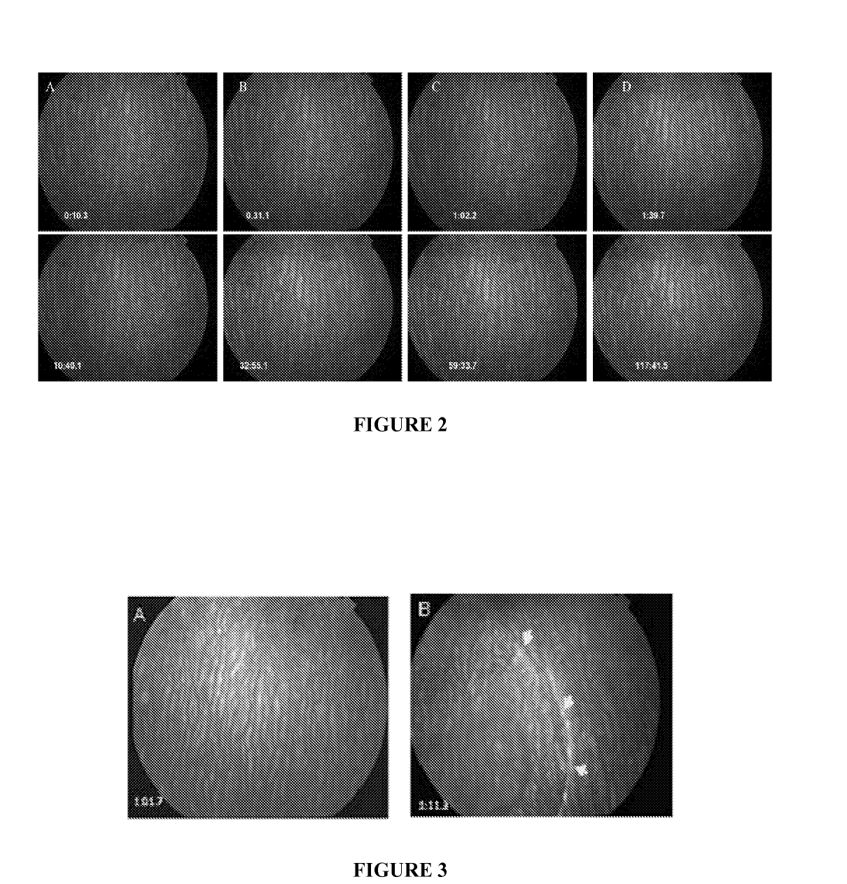

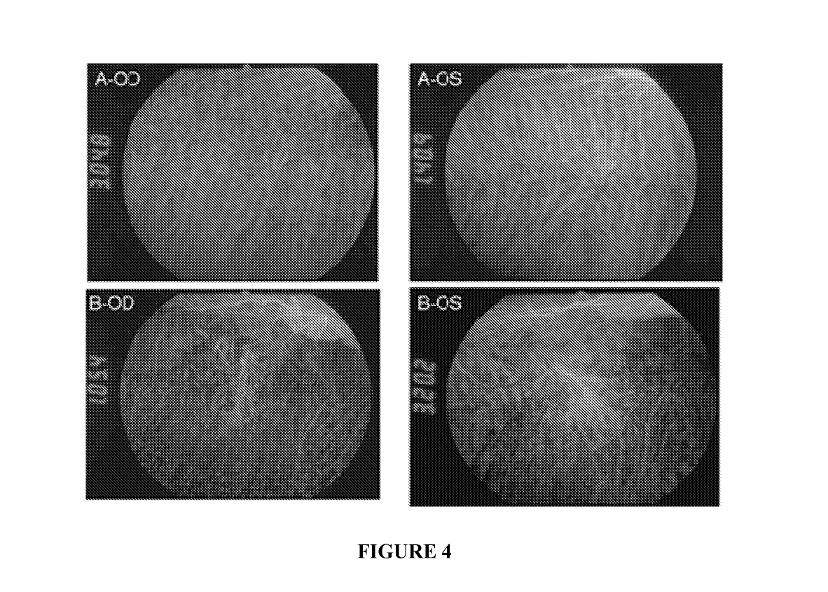

FIG. 2 shows the fluorescein images of choroidal vasculature during a 2-hours period following a single intravenous injection of FITC-dextran solution. The images from A to H in FIG. 2 show the normal choroidal angiograms captured at the time points of 10^{th} second, 30^{th} second, 1^{st} min, 1.4^{th} min, 10.4^{th} min, 32.6^{th} min, 59.3^{rd} min, and 119.4^{th} min, after intravenous administration of FITC-dextran correspondently.

FITC-dextran fundus angiography is able to provide the effective image duration up to 2 hours following a single dose intravenous injection of FITC-dextran solution.

### Example 3

Application of FITC-Dextran Fundus Angiography in the Eye with Retinal Detachment in Rabbit

Materials and Methods: The retinal detachment was induced by sub-retinal injection of Healon in the eye of pigmented rabbit. FITC-dextran with average molecular weight of 70 kDa (FD-70S, Sigma, St. Louis, Mo.) was dissolved in injection water at final concentration of 25 mg/ml. The volume of 0.3 ml of the solution was injected intravenously through the marginal vein of the rabbit. The angiograms of choroidal were obtained using a regular fluorescence fundus system (Topcon 50 VT).

The fundus angiograms of the eye with retinal detachment were obtained by FITC-dextran fundus angiography. The yellow arrows in FIG. 3 indicate the area of the retinal detachment with the clear edge of the detachment, the fluid accumulated in the sub-retinal space of the retinal detachment, and the choroidal vasculature underneath of the retinal detachment area.

FITC-dextran fundus angiography is able to provide the super clear and high resolution angiograms with the details of the edges of the detachment, the fluid accumulated in the sub-retinal space of the detachment, and the choroidal vasculature beneath the retinal detachment area.

### Example 4

FITC-Dextran Fundus Angiography on the Eye with Choroidal Neovascularization (CNV) in the Rabbit

Materials and Methods: The choroidal neovascularization (CNV) was induced by sub-retinal injection of vascular growth factors in the eye of pigmented rabbit. For FITC-dextran fundus angiography, FITC-dextran with average molecular weight of 70 kDa (FD-70S, Sigma, St. Louis, Mo.) was dissolved in injection water at final concentration of 25 mg/ml. The volume of 0.3 ml of the solution was injected through marginal vein of the rabbit. The fundus angiograms were obtained using a regular fluorescence fundus system (Topcon 50 VT).

The images of A-OD and A-OS represent the fundus angiograms from both the right eye and the left eye correspondently before CNV induction. The images of B-OD and B-OS present the fundus angiograms from both, the right eye and the left eye correspondently, with newly grown blood vessels which were indicated by red arrows.

FITC-dextran fundus angiography is able to visualize the new blood vessels grown from choroidal in the eye of rabbit and provides the angiograms with superior clarity and high resolution.

### Example 5

Evaluation of the Permeability of Choroidal Neovascularization (CNV) using FITC-Dextran Fundus Angiography

Materials and Methods: The CNV was induced by sub-retinal injection of vascular growth factor(s) in the eyes of pigmented rabbits. The FITC-dextrans with average molecular weights of 4 kDa, 20 kDa, 40 kDa, 70 kDa, 150 kDa, and 200 kDa (Sigma, St. Louis, Mo.) were selected for this test. The each tested FITC-dextran was dissolved in injection water to the final concentration of 25 mg/ml. The dose of 0.3 ml of the FITC-dextran solution was injected through a marginal vein. The fundus angiograms were obtained using a regular fluorescence fundus system (Topcon 50 VT).

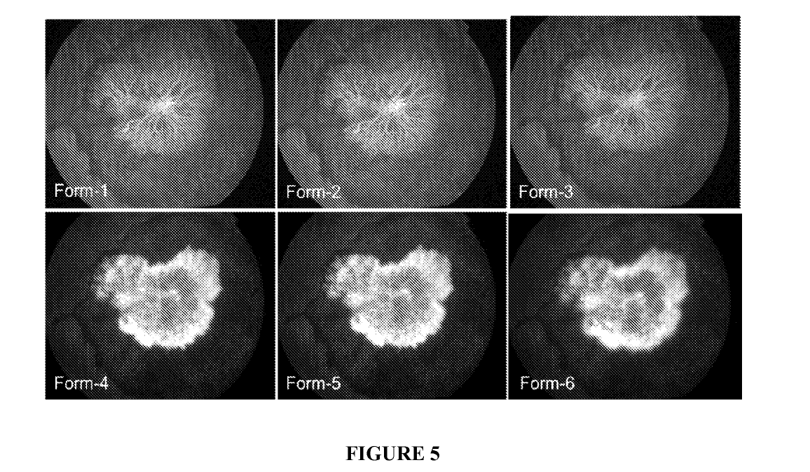

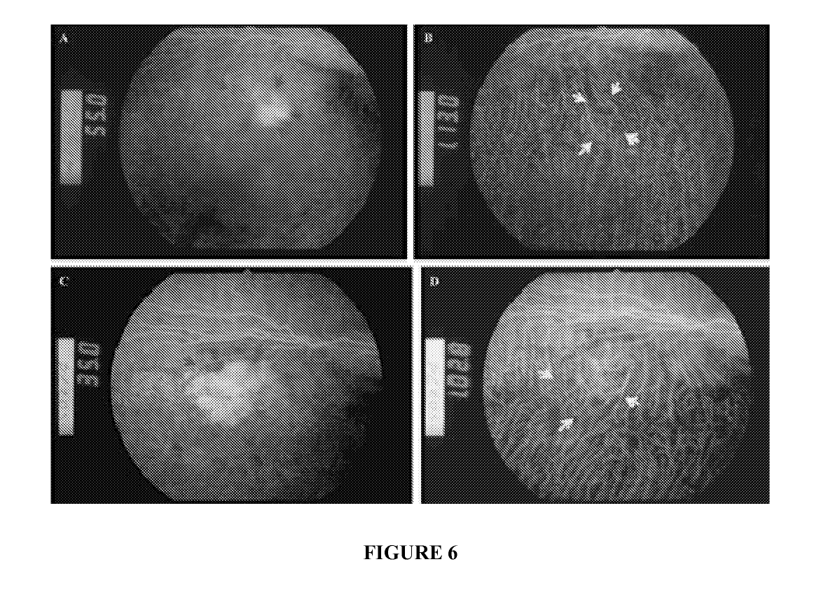

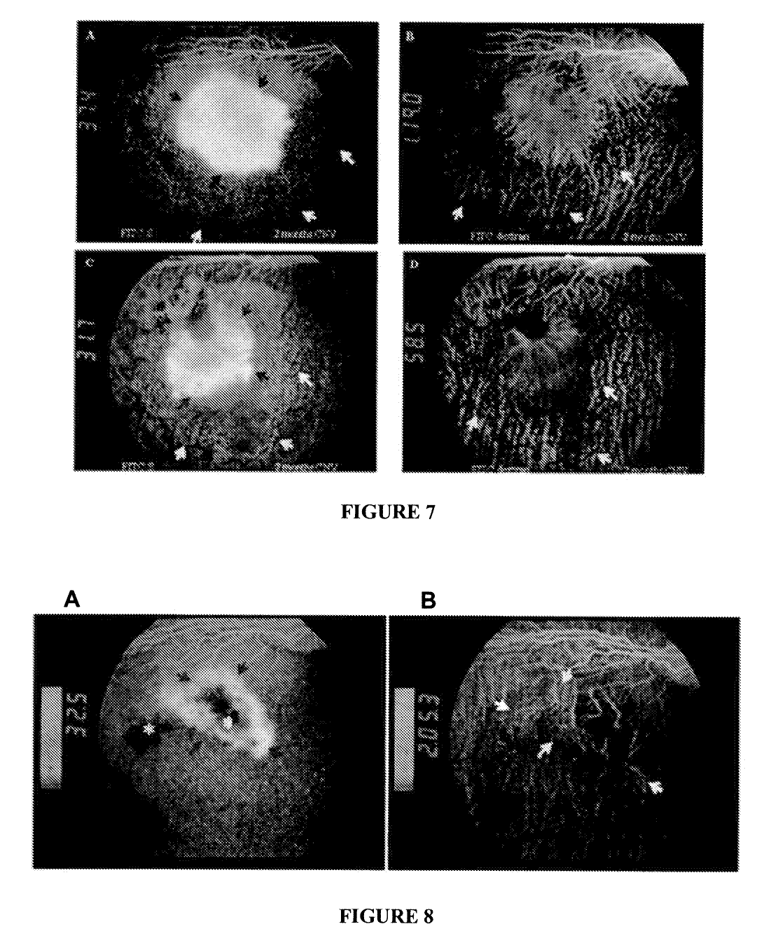

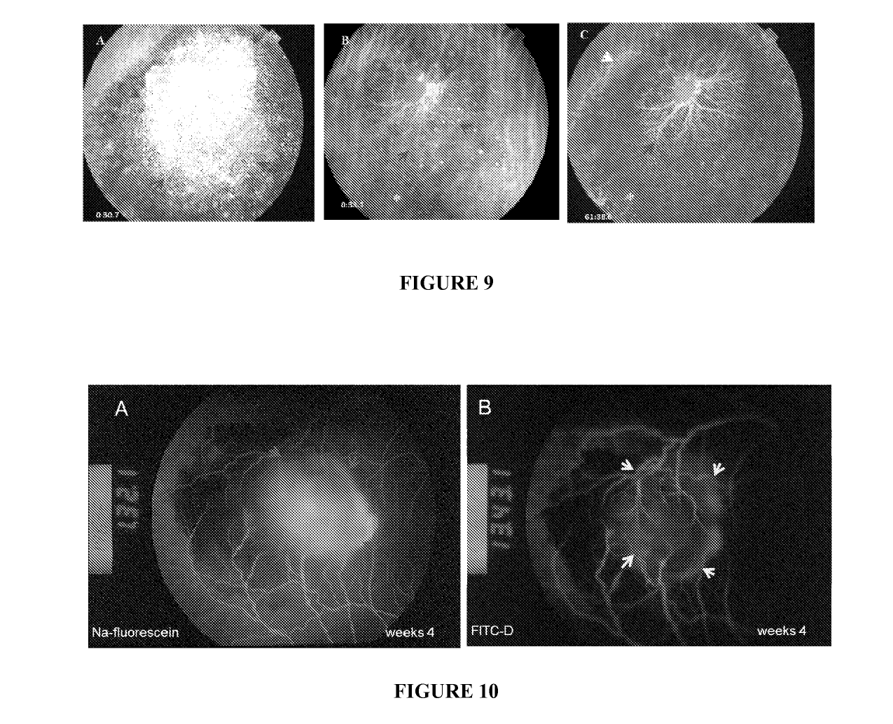

All the tested FITC-dextrans were able to visualize the CNV under the same fundus fluorescence angiography conditions. The leakage of the FITC-dextran from CNV was determined based on the density of the fluorescence in the area surrounding the CNV. The degrees of the leakage of FITC-dextrans from CNV were found to be molecular weight dependent (FIG. 5A to 5C) with the highest degree of the leakage from the FITC-dextran of 4 kDa (FIG. 5A). The degrees of the leakage were gradually reduced as the molecular weights of FITC-dextran increase. There was no visible difference among the degrees of the leakages of the FITC-dextrans with the average molecular weights of 40 kDa, 70 kDa (FIG. 5D), 150 kDa (FIG. 5E), and 20,000 kDa (FIG. 5F).

The degree of the leakage of choroidal newly grown blood vessels can be evaluated by FITC-dextran angiography using FITC-dextran with different molecular weight. In this study the degree of the leakage of FITC-dextran from choroidal blood vessel was found to be molecular weight dependent among FITC-dextran molecular weight of 4 kDa, 20 kDa, 40 kDa, and 70 kDa, with the FITC-dextran of 4 kDa has the highest detective sensitivity. There is no apparent difference in the degree of leakage of the CNV among the angiograms of 70 kDa, 150 kDa, or 2000 kDa.

### Example 6

Comparison of FITC-Dextran Fundus Angiography with Na-Fluorescein Fundus Angiography on the eyes with Choroidal Neovascularization (CNV) in rabbit

Materials and Methods: The choroidal neovascularization (CNV) in rabbit eye was induced by sub-retinal injection of vascular growth factors (VGF). For Na-fluorescence angiography, the 0.5% of Na-fluorescein solution was injected through marginal vein of the rabbit at the dose of 5 mg/kg of body weight. For FITC-dextran angiography, 0.25% solution of FITC-dextran of 70 kDa was injected through the marginal vein of the rabbit at the dose of 2.5 mg/kg of the body weight.

FIGS.-6A and -6C show the images of Na-fluorescein fundus angiography on which the area with newly grown blood vessel was presented as a hard spot of fluorescein (red arrows) without the morphological detail of the CNV. FIGS.-6B and -6D show the images of FITC-dextran angiography on which the detailed morphological information of the newly grown vasculature was revealed, including the source of the newly grown blood vessels stemmed from choroidal and the structure and the distributions of the newly grown blood vessels.

FITC-dextran fundus angiography is capable of providing detailed morphological information about the CNV which is superior over the traditional Na-fluorescein angiography.

### Example 7

Comparison of FITC-dextran Fundus Angiography with Na-Fluorescein Fundus Angiography on the Eye with Choroidal Neovascularization Complicated with Retinal Detachment

Materials and Methods: the choroidal neovascularization in the eye of rabbit was induced by sub-retinal injection of vascular growth factors (VGF). The complicated retinal detachment in the eye was caused by leakage of blood from the choroidal newly grown blood vessels and accumulated in the space of the sub-retinal between the detached retinal and the pigmented layer. For Na-fluorescence angiography, the 0.5% of Na-fluorescein solution was injected through marginal vein of the rabbit at the dose of 5 mg/kg of the body weight. For FITC-dextran angiography, the 0.25% solution of FITC-Dextran of 70 kDa was injected through the marginal vein of the rabbit at dose of 2.5 mg/kg of the body weight. The Na-fluorescence fundus angiography and the FITC-dextran fundus angiography were performed on the same rabbit with one-day interval between these two fundus angiographies.

FIGS.-7A and -7C show the images of Na-fluorescein fundus angiography from rabbit eyes with choroidal neovascularization (CNV) complicated with retinal detachment. The angiograms present the area with newly grown blood vessel as a hard spot of fluorescein (red arrows) without the morphological details of the CNV and the detachment. FIGS.-7B and -7D show the images of FITC-dextran angiography on which the morphological nature of the newly grown vasculature and the detachment of the retina were visualized with the details of the structure and the distributions of the newly grown blood vessels, the size and the edges of the detached retinas, and the choroidal blood vessels underneath the detachment.

FITC-dextran fundus angiography is capable of visualizing the newly grown vasculature and the detached retina with the details of the structure and the distributions of the newly grown blood vessels, the size and the edges of the detached retinas, and the choroidal blood vessels underneath the detachment. Such information is not available from Na-fluorescein fundus angiography.

### Example 8

Comparison of FITC-dextran Fundus Angiography with Na-Fluorescein Fundus Angiography on the Eye with Choroidal Neovascularization Complicated with Sub-retinal Hemorrhage

Materials and Methods: the choroidal neovascularization in the rabbit eye was induced by sub-retinal injection of vascular growth factors (VGF). The sub-retinal hemorrhage was caused by the pathological changes of the newly grown blood vessels underneath of the retinal. For Na-fluorescence angiography, the 0.5% of Na-fluorescein solution was injected through marginal vein of the rabbit at the dose of 5 mg/kg of the body weight. For FITC-dextran angiography, the 0.25% solution of FITC-dextran of 70 kDa was injected through the marginal vein of the rabbit at the dose of 2.5 mg/kg of the body weight. The Na-fluorescence fundus angiography and the FITC-dextran fundus angiography, were performed on the same rabbit with one-day interval between these two fundus angiographies.

FIG.-8A shows the image of Na-fluorescein fundus angiography from rabbit eye with choroidal neovascularization (CNV) complicated with retinal hemorrhage. The angiogram presents the area with newly grown blood vessels complicated with retinal hemorrhage as a hard spot of fluorescein (red arrows), without the morphological details of the CNV and the hemorrhage. FIG.-8B shows the image of FITC-dextran fundus angiography which is able to provide the morphological nature of the newly grown vasculature complicated with retinal hemorrhage (white arrows) with the details of the structures and the distributions of the newly grown blood vessels and the source of the hemorrhage.

### Example 9

Comparison of FITC-dextran Fundus Angiography with Na-Fluorescein Fundus Angiography and ICG on the Eye with CNV Complicated with Proliferated Retinopathy and Atrophy of Choroidal Circulation

Materials and Methods: the choroidal neovascularization in the eye of Dutch-belted rabbit was induced by sub-retinal injection of vascular growth factors (VGF). The proliferated retinopathy of the choroidal neovascularization membrane and the atrophy of choroidal circulation were the complications of the CNV, occurring at the late stage of the disease. For Na-fluorescence fundus angiography, the 0.5% of Na-fluorescein solution was injected through marginal vein of the rabbit at the dose of 5 mg/kg of the body weight. For FITC-dextran fundus angiography, the 0.25% solution of FITC-dextran of 70 kDa was injected through the marginal vein of the rabbit at the dose of 2.5 mg/kg of the body weight. The images of the fluorescein angiograms were obtained using a regular fluorescence fundus system (Topcon 50 VT). For ICG fundus angiography, the 0.25% solution of ICG was injected through the marginal vein of the rabbit at the dose of 2.5 mg/kg of the body weight. The fundus angiograms of IGC were obtained using the fundus image system (Topcon 50 VT) at wavelength of 800 nm. The angiographs of Na-fluorescein, ICG, and FITC-dextran were performed on the same rabbit eye with one-day internal between each angiography.

FIG.-9A shows the angiogram of the Na-fluorescein fundus angiography from the eye with CNV complicated with proliferated retinopathy and atrophy of the choroidal circulation. On the angiogram the area of CNV was present as a high density fluorescence spot (red arrows in 9A) without morphological details of the CNV and the proliferated retinopathy and the atrophy of choroidal circulation. FIG.-9B presents the angiogram from ICG fundus angiography of the same eye. The angiogram of ICG is able to visualize the structure of the CNV and the injured choroidal vascular networks (red arrows in B). FIG.-9C presents the angiogram of FITC-dextran fundus angiography from the same eye. Comparing with ICG-angiography or Na-fluorescein angiography, FITC-dextran angiography is able to visualize the newly grown blood vessels with details of the CNV and changes of the proliferated retinopathy and atrophy in the choroidal vasculature.

FITC-dextran fundus angiography shows the advantages over the Na-fluorescein fundus angiography and ICG fundus angiography on the visualizing CNV complicated with proliferated retinopathy and atrophy of choroidal circulation.

Table 1 shows the summary of the comparison of Na-fluorescein fundus angiography with ICG fundus angiography and FITC-dextran fundus angiography on the eye with proliferative CNV.

TABLE 1

| | Na- | | FITC- |
|---|---|---|---|
| | Fluorescein | ICG | Dextran |
| Neovascularization | | | |
| Visualization | | | |
| Normal retinal vessels | + | − | + |
| Retinal neovascularization | + | − | + |
| Normal choroidal vessels | − | + | + |
| Choroidal neovascularization | − | + | + |
| Duration: | | | |
| Normal retinal vessels | 2-5 minutes, | − | >1 hour |
| | then watch out | | |
| Retinal neovascularization | up to | − | >1 hour |
| | 10 seconds | | |
| | then leak out | | |
| Normal Choroidal | − | 3-5 minute | >1 hour |
| vasculatures | | | |
| Choroidal neovascularization | − | 3-5 minute | >1 hour |
| Angiography blocked by | − | − | + |
| hemorrhage | | | |
| Clarity of angiography | good | poor | best |
| Neovascularization complex | poor | good | best |
| equipment | Fluorescein | infrared | Fluorescein |
| | capability | capability | capability |
| RPE toxicity | − | + | − |

### Example 10

Comparison of FITC-Dextran Fundus Angiography with Na-Fluorescein Fundus Angiography on the Eye with Retinal Neovascularization in Monkey

Materials and Methods: Retinal neovascularization in the eye of the monkey was induced by sub-retinal administration of vascular growth factors (VGF). For Na-fluorescence fundus angiography, the 0.5% of Na-fluorescein solution was injected through the antecubital vein at the dose of 5 mg/kg of the body weight. For FITC-dextran fundus angiography, the 0.25% solution of FITC-dextran 40 kDa was injected through the antecubital vein at the dose of 2.5 mg/kg of the body weight. The fundus angiograms were obtained using a regular fluorescence fundus system (Topcon 50 VT). The FITC-dextran fundus angiography and the Na-fluorescein fundus angiography were performed on the same eye of the monkey with one-day interval between the two fundus angiographies.

FIG.-10A presents the fundus angiogram of Na-fluorescein from the eye with CNV in monkey. On the angiogram the area of CNV was visualized as a high density fluorescence spot (arrows in 10A) without the morphological details of the CNV. FIG.-10B presents the fundus angiogram of FITC-dextran from the same eye. The angiogram of FITC-dextran is able to provide the morphological details of the newly grown blood vessels including the source of the newly grown blood vessels and its distribution and the status of the leakage from the newly grown blood vessels.

FITC-dextran fundus angiography shows the advantages over the Na-fluorescein fundus angiography on visualizing the retinal newly grown blood vessels in the eye of monkey.

### Example 11

FITC-dextran Fluorophotometry Analysis on the Eye with Endotoxin-induced Uveitis in Rabbit (EIU)

Materials and Methods: Endotoxin-induced uveitis was induced by intravenous injection of lipopolysaccharide (LPS) in the rabbit which results in the breakdown of the blood-eye barrier due to the inflammatory response. The degree of the blood-aqueous barrier breakdown in the eyes with uveitis was evaluated by measuring the fluorescein levels in the anterior and the posterior segments of the eye using fluorophotometry at the 90th minutes following LPS challenge. The FITC-dextran of 70 kDa, at dose of 3 mg/kg was given at the 5^{th} minutes before fluorescence measurement. The fluorescence levels in the anterior and the posterior of the eye were then quantified for the evaluation of the degree of intraocular inflammation.

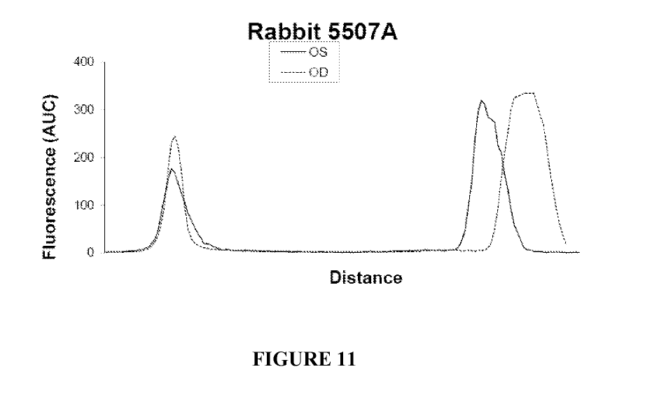

Two fluorescence peaks from anterior chamber and posterior segment of the eye from fluorescence measurement were plotted on FIG. 11. The peak on the right in FIG. 11 represents the fluorescein level in the anterior chamber and the peak on the left in FIG. 11 represents the fluorescein level in the posterior segment of the eye. The size of the area under curve (AUC) of the peaks represents the degree of intraocular inflammation.

FITC-dextran can be used as a tracer of fluorophotometry analysis for the quantitative evaluation of the leakage of intraocular vasculatures in the eye with endotoxin-induced uveitis (EIU).

### REFERENCES

Atkinson E G, Jones S, et al: Molecular size of retinal Vascular Leakage Determined by FITC-Dextran Angiography in Patients with Posterior Uveitis. Eye (1991) 5: 440-446.

Baffi J, Byrnes G, et al: Choroidal neovascularization in the rat induced by adenovirus mediated expression of vascular endothelial growth factor. Invest Ophthalmol Vis Sci (2000) 41:3582-3589.

Barreiro, R., Schadlu R. et al: The role of Fas-FasL in the development and treatment of ischemic retinopathy. Invest Ophthalmol Vis Sci (2003) 44(3): 1282-1286.

Bee Y S, Sheu S J, et al: Topical application of recombinant calreticulin peptide, vasostatin 48, alleviates laser-induced choroidalal neovascularization in rats; Molecular Vision (2010) 16:756

Bischoff P M, and Flower, R W: Ten years' experience with choroidalal angiography using indocyanine green dye: a new routine examination or an epilogue? Doc Ophthalmol (1985) 60: 235-291.

Campa C., Kasman I. et al. Effects of an anti-VEGF-A monoclonal antibody on laser-induced choroidalal neovascularization in mice: optimizing methods to quantify vascular changes. Invest Ophthalmol Vis Sci (2008) 49(3): 1178-1183.

Campos M, Amaral J, et al. A novel imaging technique for experimental choroidalal neovascularization. Invest Ophthalmol Vis Sci (2006) 47(12): 5163-5170.

D'Amato R, Wesolowski E, et al: Microscopic visualization of the retina by angiography with high-molecular-weight fluorescein-labeled dextrans in the mouse. Microvasc. Res (1993) 46, 135-142.

Davies M H, Eubanks J P, et al. Increased retinal neovascularization in Fas ligand-deficient mice. Invest Ophthalmol Vis Sci (2003) 44(7): 3202-3210.

Davies M H, Eubanks J P, et al. Microglia and macrophages are increased in response to ischemia-induced retinopathy in the mouse retina. Mol Vis (2006) 12: 467-477.

Davies M H, Stempel A J et al. MCP-1 deficiency delays regression of pathological retinal neovascularization in a model of ischemic retinopathy. Invest Ophthalmol Vis Sci (2008) 49(9): 4195-4202

Diego G. Espinosa-Heidmann I S, et al: Cousins Age as an Independent Risk Factor for Severity of Experimental Choroidalal Neovascularization; Invest Ophthalmol Vis Sci. (2002) 43:1567-1573

Huang, H, Vasilakis P. et al: Parstatin suppresses ocular neovascularization and inflammation. Invest Ophthalmol Vis Sci. (2010) 51(11): 5825-5832.

Edelman J and Castro M R: Quantitative Image Analysis of Laser-induced Choroidalal Neovascularization in Rat; Exp. Eye Res. (2000) 71, 523-533.

Feeney S A, Simpson D A, et al: Role of vascular endothelial growth factor and placental growth factors during retinal vascular development and hyaloids regression. Invest Ophthalmol Vis Sci (2003) 44(2): 839-847.

Fu Y, Ponce M L. et al: Angiogenesis inhibition and choroidalal neovascularization suppression by sustained delivery of an integrin antagonist, EMD478761. Invest Ophthalmol Vis Sci (2007) 48(11): 5184-5190.

Funakoshi T., Birsner A E, et al: Antiangiogenic effect of oral 2-methoxyestradiol on choroidalal neovascularization in mice. Exp Eye Res (2006) 83(5): 1102-1107.

Giuseppe Q, Francesco P et al: Retinal toxicity of indocyanine green. Int Ophthalmol (2008) 28:115-118.

Gelin L E: Studies in Anemia of Injury, Acta Chir Scand, (1956) Suppl 210.

Gronwall A: Dextran and its Use in Colloidal Infusion Solution. Uppsala, Sweden: Almquist and Wiksell, Printers & Publishers, 1957.

Guyer, D R, Puliafito, C P, et al: Digital indocyanine green angiography in chorioretinal disorders, Ophthalmology (1992) 99: 287-290.

Honda, M., Asai T., et al: Suppression of choroidalal neovascularization by intravitreal injection of liposomal SU5416. Arch Ophthalmol. (2011) 129(3): 317-321.

Hu W, Jiang A, et al: Expression of VLDLR in the retina and evolution of subretinal neovascularization in the knockout mouse model's retinal angiomatous proliferation. Invest Ophthalmol Vis Sci (2008) 49(1): 407-415.

Ida H., Tobe T, et al: RPE cells modulate subretinal neovascularization, but do not cause regression in mice with sustained expression of VEGF. Invest Ophthalmol Vis Sci (2003) 44(12): 5430-5437.

Kim, Y H, Chung I Y, et al: Triamcinolone suppresses retinal vascular pathology via a potent interruption of proinflammatory signal-regulated activation of VEGF during a relative hypoxia. Neurobiol Dis (2007) 26(3): 569-576.

Kinose, F., Roscilli G, et al: Inhibition of retinal and choroidalal neovascularization by a novel KDR kinase inhibitor. Mol Vis (2005) 11: 366-373.

Lightman S L, Caspers-Velu L E et al: Angiography with fluorescein-labeled dextrans in a primate model of uveitis. Arch Ophhalmol. (1987) 105: 844-848.

McKenna, D J., Simpson D A, et al: Expression of the 67 kDa laminin receptor (67LR) during retinal development: correlations with angiogenesis. Exp Eye Res (2001) 73(1): 81-92.

McMenamin P G: Optimal methods for preparation and immunostaining of iris, ciliary body, and choroidalal whole mounts. Invest Ophthalmol Vis Sci. (2000) 41(10):3043-3048.

McNaught E I, Foulds W S, et al: The permeability of the posterior blood ocular barrier after xenon photocoagulation: a study using fluorescein labeled dextrans. Br J Ophthalmol. (1981) 65:473-477.

Miller J W, Adamis A P, et al: Vascular endothelial growth factor/vascular permeability factor is temporally and spatially correlated with ocular angiogenesis in a primate model. Am J Pathol. (1994) 145:574-584.

Nambu H, Nambu R, et al: Combretastatin A-4 phosphate suppresses development and induces regression of choroidalal neovascularization. Invest Ophthalmol Vis Sci (2003) 44:3650-3655.

Ohno-Matsui, K., Uetama T, et al: Reduced retinal angiogenesis in MMP-2-deficient mice. Invest Ophthalmol Vis Sci (2003) 44(12): 5370-5375.

Olson, J L, Courtney R J, et al: Intravitreal infliximab and choroidalal neovascularization in an animal model. Arch Ophthalmol (2007) 125(9): 1221-1224.

Olson J L, Courtney R J, et al: Intravitreal anakinra inhibits choroidalal neovascular membrane growth in a rat model.” Ocul Immunol Inflamm (2009) 17(3): 195-200.

Patrycja N S, Judy R B, et al: Vascular regrowth following photodynamic therapy in the chicken embryo chorioallantoic membrane; Angiogenesis (2010) 13:281-292.

Rabkin M D, Bellhorn M B, et al: Selected molecular weight dextrans for in vivo permeability studies of rat retinal vascular disease. Exp. Eye. Res. (1977) 24: 607-612.

Ringvold A, Olsen E G: Choroidalal fluorescein angiography in rabbit. Acta Ophthalmol (Copenh). (1979) 57:513-521.

Salminen L., Chioralia G. and Kremer F: Fluorescein-labelled Dextrans as tracer substance for experimental Angiograms. Acta Ophthalmologica. (1976) 54: 665-667.

Schatz., H S, Burton, T, et al: Interpretation of fundus fluorescein angiography, St Louis, (1978) Mosby-Year Book, Inc. C. V. Mosby Co., 1978

Tan, L E, Orilla W, et al: Effects of vitreous liquefaction on the intravitreal distribution of sodium fluorescein, fluorescein dextran, and fluorescent microparticles.” Invest Ophthalmol Vis Sci (2011) 52(2): 1111-1118.

Thorsen. G and Hint H: Aggregation, Sedimentation and intravascular slugging of Erythrocytes, Acta Chir Scand, (1950) Suppl 154.

Tobe T, Okamoto N, et al: Evolution of neovascularization in mice with overexpression of vascular endothelial growth factor in photoreceptors. Invest Ophthalmol Vis Sci. (1998) 39:180-188.

Tolentino M J., Husain D, et al: Angiography of fluoresceinated anti-vascular endothelial growth factor antibody and dextrans in experimental choroidalal neovascularization. Arch Ophthalmol (2000) 118(1): 78-84.

Vaughan D, Asbury T, and Tabbara, KF: General ophthalmology, Twelfth Edition, Appleton & lange, Norwalk, Conn./San Mateo, Calif., 1989.

Wang F E, Shi G, et al: Receptor tyrosine kinase inhibitors AG013764 and AG013711 reduce choroidalal neovascularization in rat eye. Exp Eye Res (2007) 84(5): 922-933.

Wolfe J D and Csaky K G: Indocyanine green enhanced retinal vessel laser closure in rats: histologic and immunohistochemical observations. (2004) Exp Eye Res 79(5): 631-638.

Waterbury L D, and Flach A J: Comparison of Ketorolac Tromethamine, Diclofenac Sodium, and Loteprednol Etabonate in an Animal Model of Ocular Inflammation; Journal Of Ocular Pharmacology And Therapeutics, (2006) Volume 22, Number 3.

Yannuzzi, L A, Ed: Laser photocogulation of the macula. Philadelphia, JB Lippincitt Co. (1989)

Yannuzzi L A, Slakter J S, et al: Digital indocyanine green videoangiography and choroidalal neovascularization, Retina (1992) 12: 191-223.

Yu P K, Cringle S J, et al: Low power laser treatment of the retina ameliorates neovascularization in a transgenic mouse model of retinal neovascularization. Exp Eye Res (2009) 89(5): 791-800.

Zou Y, Jiang W, et al: Effect of tetramethylpyrazine on rat experimental choroidalal neovascularization in vivo and endothelial cell cultures in vitro. Curr Eye Res (2007) 32(1): 71-75.

What is claimed is:

1. A method for performing fundus angiography comprising administration of a fluorescein derivative-linked polymer as a fluorescence angiographic dye followed by observation and photography of ocular fundus circulation using a fluorescence fundus camera with an image system.

2. The method of claim 1, wherein the fluorescein derivative-linked polymer has a weight average molecular weight ranging from 3 k Daltons to 2000 k Daltons.

3. The method of claim 2, wherein the weight average molecular weight is selected from any one of the group consisting of 3 k, 5 k, 10 k, 20 k, 40 k, 70 k, 120 k, 250 k, 200 k, and 2000 k Daltons.

4. The method of claim 1, wherein the fluorescein derivative-linked polymer is a mixture of one or more fluorescein derivative-linked polymer having weight average molecular weight ranging from 3 k Daltons to 2000 k Daltons.

5. The method of claim 2, wherein the weight average molecular weight is 70 k Daltons.

6. The method of claim 1, wherein the fluorescein derivative-linked polymer is selected from a group consisting of fluorescein isothiocyanate dextrans (FITC-dextrans), NHS-fluorescein dextran, pentafluorophenyl esters dextran (PFP-dextran), Alexa 488 dextran, FluoProbes 488 dextran, DyLight 488 dextran, fluorescein isothiocyanate d-glucose polymer, fluorescein isothiocyanate chitin/its derivatives, and fluorescein isothiocyanate cellularglycan.

7. The method of claim 1, wherein the fluorescein derivative-linked polymer is FITC-dextran.

8. The method of claim 6, wherein each glucose unit of the dextrans is linked with one or more FITC moiety(s) through chemical linkage at C1, C2, C3, C4, and/or C6 carbon position of the glucose unit of the dextrans.

9. The method of claim 1, wherein the fluorescein derivative-linked polymer is dissolved in water, saline, phosphate buffered saline (PBS) or other buffered systems with or without other chemical additive(s).

10. The method of claim 9, wherein the fluorescein derivative-linked polymer is dissolved at a concentration ranged from 0.1 wt. % to 50 wt. %, preferably from 0.1 wt. % to 10 wt. %, more preferably from 0.5 wt. % to 5 wt. %.

11. The method of claim 1, wherein the fluorescein derivative-linked polymer is administrated at a dose range of 0.1 to 100 mg/kg, preferably from 1 to 50 mg/kg.

12. The method of claim 1, wherein the fluorescein derivative-linked polymer is administrated through intravenous, intravascular administration or other routes of administration based on doctor's instruction.

13. A method for diagnosis of eye diseases comprising administration of a fluorescein derivative-linked polymer as a fluorescence angiographic dye to a subject in need of receiving fundus angiography, followed by observation and photography of ocular fundus circulation using a fluorescence fundus camera with an image system.

14. The method of claim 13, wherein the eye diseases are selected from the group consisting of retinal, sub-retinal or choroidal neovascularization; proliferative diabetic retinopathy; retinopathy of prematurity; retinal tumors; degenerative conditions; ocular inflammation and optic nerve lesions; choroidal tumors; and retinal vascular occlusion.

15. A method for qualitative or quantitative analysis of changes of ocular blood vessels comprising administration of a fluorescein derivative-linked polymer as a fluorescence angiographic dye to a subject in need of receiving fundus angiography, followed by observation and photography of ocular fundus circulation using a fluorescence fundus camera with an image system.

16. The method of claim 15, wherein the eye diseases are selected from the group consisting of retinal, sub-retinal or choroidal neovascularization; proliferative diabetic retinopathy; retinopathy of prematurity; retinal tumors; degenerative conditions; ocular inflammation and optic nerve lesions; choroidal tumors; and retinal vascular occlusion.
